(19) United States
(12) Patent Application Publication
(10) Pub. No.: US 2021/0174913 A1
(43) Pub. Date: Jun. 10, 2021

(54) METHOD, APPARATUS AND STORAGE MEDIUM FOR LABELING CAPSULE ENDOSCOPY REPORT

(71) Applicants: ANKON TECHNOLOGIES CO., LTD, Wuhan (CN); ANX IP HOLDING PTE. LTD., Singapore (SG)

(72) Inventors: Wenjin Yuan, Wuhan (CN); ZHIWEI HUANG, WUHAN (CN); Hao ZHANG, WUHAN (CN); HANG ZHANG, Wuhan (CN)

(73) Assignees: ANKON TECHNOLOGIES CO., LTD, Wuhan (CN); ANX IP HOLDING PTE. LTD., SINGAPORE (SG)

(21) Appl. No.: 17/112,976

(22) Filed: Dec. 4, 2020

(30) Foreign Application Priority Data
Dec. 6, 2019 (CN) 201911242144.7

(51) Int. Cl. G16H 10/40; G06F 16/24; G06F 16/28

(52) U.S. Cl. CPC G16H 10/40; G06F 16/28; G06F 16/24

(57) ABSTRACT

The present invention discloses a method, an apparatus and a storage medium for labeling capsule endoscopy report. The method includes: collecting p report samples to establish an initial corpus database; parsing the report samples in the initial corpus database, to establish a named entity recognition dictionary and a pattern rules database, and removing duplicate texts from the named entity recognition dictionary and the pattern rules database; since the q-th report sample is collected, q=p+1, querying the named entity recognition dictionary and pattern rules database with texts appearing in the report sample, to automatically label the current report sample. The present invention can build a database by parsing a small number of labeled report samples, make subsequent report samples query the database using specific rules, and then label the report samples automatically in a fast and effective manner, save labor costs and improve labelling efficiency.

### CROSS-REFERENCE OF RELATED APPLICATIONS

The application claims priority to Chinese Patent Application No. 201911242144.7 filed on Dec. 6, 2019, the contents of which are incorporated by reference herein.

### FIELD OF INVENTION

The present invention relates to the field of medical device, and more particularly to a method, an apparatus and a storage medium for labelling a capsule endoscopy report.

### BACKGROUND

Capsule endoscope is a medical device that integrates core components such as a camera and a wireless transmission antenna into a capsule that can be swallowed by a subject. As swallowed into the body of the subject, the capsule endoscope takes images in the digestive tract while transmitting the images to the outside of the body for review and evaluation by a physician.

Once a capsule endoscopy is completed, an examination report is generated, including findings, diagnosis results, and recommendations. Due to the different habits and writing styles of each doctor, each report is different. Also, because of the small number of GI doctors and their heavy workload, omissions and mistakes may be caused in the report. In order to facilitate subsequent review and analysis, it is usually necessary to organize and label the report, to form structured data.

In the prior art, manual labelling is usually used to organize examination reports, which wastes manpower and increases labelling costs.

### SUMMARY OF THE INVENTION

The present invention discloses a method, an apparatus and a storage medium for labelling a capsule endoscopy report.

It is one object of the present invention to provide a method for labelling a capsule endoscopy report, the method comprising:

step S1, collecting p report samples to establish an initial corpus database, any of the p report samples comprising an original text and labeled information, and the labeled information is a naming category corresponding to each noun in the original text;

step S2, parsing the report samples in the initial corpus database, to establish a named entity recognition dictionary and a pattern rules database, and removing duplicate texts from the named entity recognition dictionary and the pattern rules database;

wherein the named entity recognition dictionary comprises named categories in the report samples and nouns corresponding to each named category, and the pattern rules database comprises unrecognized texts in the report samples and rules, laws, and characteristics corresponding to the unrecognized texts;

step S3, since the q-th report sample is collected, q=p+1, querying the named entity recognition dictionary and pattern rules database with texts appearing in the report sample, to automatically label the current report sample.

In an embodiment of the present invention, after step S3, the method further comprises:

step S4, reviewing the automatically labeled report sample, revising errors when there are errors in the automatically labeled report sample, transferring the revised report sample to the original corpus database, and re-iterating and updating the named entity recognition dictionary and pattern rules database; identifying that the labelling of the current report sample completes when there are no errors in the automatically labeled report sample.

In an embodiment of the present invention, step S2 specifically comprises: segmenting each report sample into a plurality of short sentences by punctuation and storing the first obtained short sentences to form a statement database.

In an embodiment of the present invention, in the process of establishing the statement database in step S2, the method further comprises: parsing each obtained short sentence, and determining whether the current short sentence already exists in the statement database; omitting to process the current short sentence when the current short sentence already exists in the statement database, adding the current short sentence to the statement database when the current short sentence does not exist in the statement database;

parsing the statement database, to establish a named entity recognition dictionary and a pattern rules database, and removing duplicate texts from the named entity recognition dictionary and the pattern rules database.

In an embodiment of the present invention, the step S2 further comprises:

creating a prefix dictionary according to the named entity recognition dictionary, the prefix dictionary storing noun groups corresponding to each noun in the named entity recognition dictionary;

when the named entity recognition dictionary is composed of {d₁, . . . ,dᵢ, . . . ,dₙ}, any noun group in the prefix dictionary is expressed as: {d_{i_1}, . . . ,d_{i_j}, . . . ,d_{i_Li}};

wherein, n denotes the total number of nouns in the named entity recognition dictionary, dᵢ denotes the i-th noun in the named entity recognition dictionary, i [symbol] 1, 2 . . . n, the i-th noun comprises Li characters arranged in sequence, d_{i_j} denotes the word consisting of the characters from the 1st one to the j-th one arranged in sequence, j [symbol] 1, 2 . . . Li;

traversing the prefix dictionary and keeping only one of the same words;

the step S3 specifically comprises: since the q-th report sample is collected, querying the named entity recognition dictionary, prefix dictionary and pattern rules database with the texts appearing in the report sample, to automatically label the current report sample.

In an embodiment of the present invention, the step S3 further comprises:

segmenting each report sample into a plurality of short sentences by punctuation when the q-th report sample is collected;

querying the prefix dictionary with word x_{t_k} formed from the t-th character to the k-th character in each short sentence, the value of t is [1,XN], the value of k is [t,XN], wherein XN is the total number of characters in current short sentence;

determining whether x_{t_k} exists in the prefix dictionary, taking t=1 for the first time of determination,

taking k=k+1 when x_{t_k} exists in the prefix dictionary, continuing to determine whether x_{t_k+1} exists in the prefix dictionary, till the x_{t_k+1} is not in the prefix dictionary, then querying the named entity recognition dictionary using x_{t_k} as the keyword, and when a noun corresponding to the keyword is found, labeling the current noun with the naming category of the found noun, and when the noun corresponding to the keyword is not found, doing greedy matching for current word x_{t_k} and labelling according the matching result;

when the noun corresponding to the current word x_{t_k} is still not found by greedy matching, giving up labeling with querying the named entity recognition dictionary as the standard.

In an embodiment of the present invention, “when the noun corresponding to the keyword is not found, doing greedy matching for current word x_{t_k} and labelling according the matching result” in step S3 specifically comprises:

doing a forward greedy matching for the current word x_{t_k};

in the process of forward greedy matching, keeping k=k−1, and each time k is re-assigned, querying the named entity recognition dictionary using x_{t_k-1} as keyword, and when the corresponding noun is found, labelling the current noun with the naming category of the found noun, and when the corresponding noun is still not found when k=t, performing backward greedy matching for the word x_{t_k};

in the process of backward greedy matching, keeping t=t+1, and each time t is re-assigned, querying the named entity recognition dictionary using x_{t+1_k} as keyword, and when the corresponding noun is found, labelling the current noun with the naming category of the found noun, and when the corresponding noun is still not found when t=k, determining that the combination in any sequence of characters from the t-th one to the k-th one in the current word is not successfully queried in the named entity recognition dictionary.

In an embodiment of the present invention, in the process of querying the named entity recognition dictionary, prefix dictionary and pattern rules database with the texts appearing in the report sample in step S3, the method further comprises:

first querying the named entity recognition dictionary with the texts appearing in the report sample, and continuing to query the pattern rules database with the texts appearing in the report sample when no corresponding text is found in the named entity recognition dictionary.

It is another object of the present invention, to provide an electronic device comprising a memory and a processor. The memory stores a computer program that runs on the processor, and the processor executes the program to implement the steps of method for labelling the capsule endoscopy report described above.

It is still another object of the present invention, to provide a computer-readable storage medium for storing a computer program. The computer program is executed by the processor to implement the steps of method for labelling the capsule endoscopy report described above.

Compared with the prior art, the present invention has the advantages including building a database by parsing a small number of labeled report samples, making subsequent report samples query the database using specific rules, and then labelling the report samples automatically in a fast and effective manner, saving labor costs and improving labeling efficiency.

### BRIEF DESCRIPTION OF THE DRAWINGS

FIG. 1 is a schematic flowchart of a method for labelling a capsule endoscopy report, in accordance with an embodiment of the present invention.

FIG. 2 is a schematic flowchart of a preferred embodiment of the method for labelling the capsule endoscopy report developed on the basis of FIG. 1.

FIG. 3 is a schematic flowchart of a specific implementation process of one of the steps in FIG. 1.

### DETAILED DESCRIPTION

The present invention can be described in detail below with reference to the accompanying drawings and preferred embodiments. However, the embodiments are not intended to limit the invention, and the structural, method, or functional changes made by those skilled in the art in accordance with the embodiments are included in the scope of the present invention.

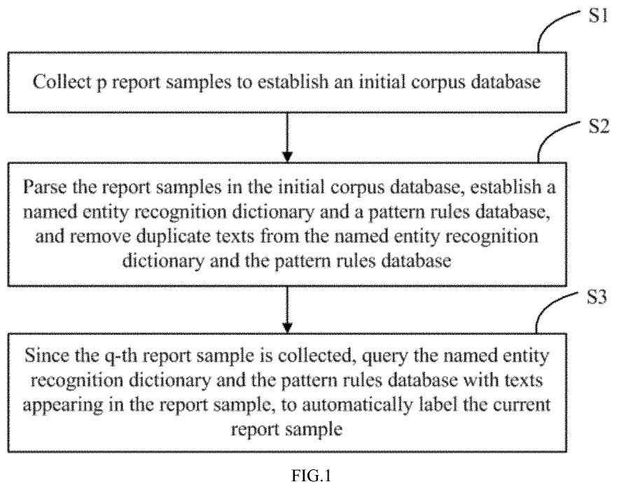

Referring to FIG. 1, the first embodiment of the present invention provides a method for labelling a capsule endoscopy report, the method comprising:

step S1, collecting p report samples to establish an initial corpus database. Any of the p report samples comprises an original text and labeled information, and the labeled information is a naming category corresponding to each noun in the original text;

step S2, parsing the report samples in the initial corpus database, establishing a named entity recognition dictionary and a pattern rules database, and removing duplicate texts from the named entity recognition dictionary and the pattern rules database;

wherein the named entity recognition dictionary comprises named categories in the report samples and nouns corresponding to each named category, and the pattern rules database comprises unrecognized texts in the report samples and rules, laws, and characteristics corresponding to the unrecognized texts;

step S3, since the q-th report sample is collected, q=p+1, querying the named entity recognition dictionary and the pattern rules database with texts appearing in the report sample, to automatically label the current report sample.

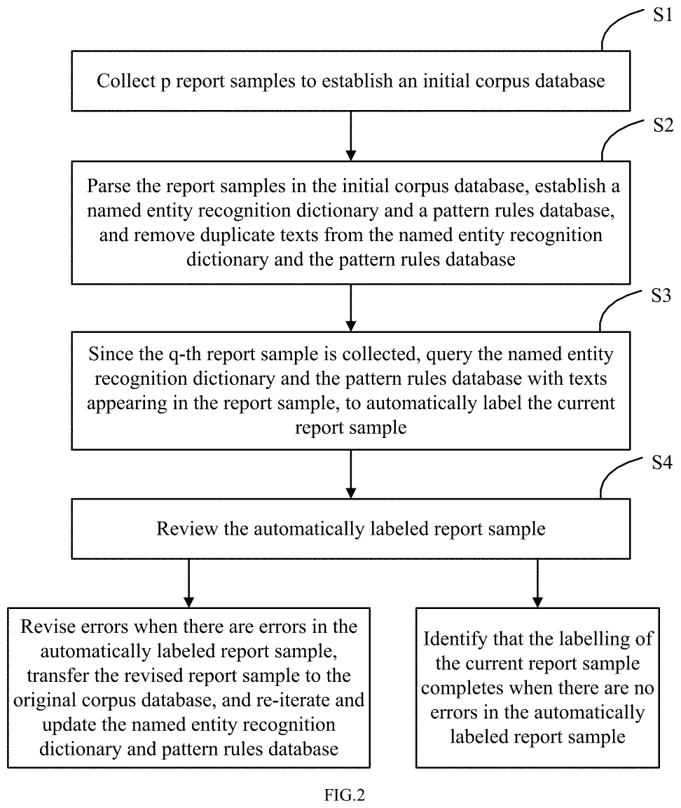

Referring to FIG. 2, in a preferred embodiment of the present invention, after step S3, the method further comprises:

step S4, reviewing the automatically labeled report sample, revising errors when there are errors in the automatically labeled report sample, transferring the revised report sample to the original corpus database and re-iterating and updating the named entity recognition dictionary and pattern rules database; identifying that the labelling of the current report sample completes when there are no errors in the automatically labeled report sample.

Further, in the process of querying the named entity recognition dictionary and the pattern rules database with the texts appearing in the report sample in step S3, the method further comprises: first querying the named entity recognition dictionary with the texts appearing in the report sample, and continuing to query the pattern rules database with the text appearing in the report sample when no corresponding text is found in the named entity recognition dictionary.

In the specific implementation process of the present invention, due to the large number of nouns contained in the report samples, the cost of manual labeling is relatively high. Therefore, in step S1, only P copies of a large number of report samples are selected and labeled manually. In subsequent steps, other report samples are labeled automatically using a gradual and iterative method.

For step S2, each report sample comprises a large number of texts. In the preferred embodiment of the present invention, in order to reduce the amount of data to be processed, in the process of parsing the report samples in the initial corpus database, P report samples are split into sentences for storage for subsequent recall. Also, because there are too many report samples, report samples, descriptions of the same naming categories in the report samples, and the sentences after the report samples are split may be repeated in large numbers, so the overlapping texts are de-duplicated at the same time in the process of building the following statement database. Specifically, step S2 specifically comprises: segmenting each report sample into a plurality of short sentences by punctuation and storing the first obtained short sentences to form a statement database; in the process of establishing the statement database, parsing each obtained short sentence, and determining whether the current short sentence already exists in the statement database, when the current short sentence already exists in the statement database, omitting to process the current short sentence, when the current short sentence does not exist in the statement database, adding the current short sentence to the statement database; parsing the statement database, to establish a named entity recognition dictionary and a pattern rules database, and removing duplicate texts from the named entity recognition dictionary and the pattern rules database.

In the process of building the statement database, the information stored is the sentences obtained by segmenting the report sample, as well as the labeled information corresponding to each noun in each sentence, and the same sentence is collected and recorded only once, thus reducing the amount of data and speeding up the building of the statement database.

In an embodiment of the present invention, the value of P can be specifically set as needed. In a specific example, the value range of p is given as [50, 5000].

Further, by parsing the statement database, the nouns included in each sentence and the labeled information corresponding to the nouns can be obtained.

In a specific example of the present invention, since this method is usually used for labeling report samples generated after capsule endoscopy, the naming categories comprise: organ identification, disease type, etc. In other applications of the present invention, the type and specific content of the naming category can also be specifically set as required. In this specific example, the nouns corresponding to the organ identification are usually digestive tract organs and anatomical structures, such as: esophagus, stomach, antrum, etc., and the nouns corresponding to the disease type are cancer, tumor, polyp, ulcer, etc.

In the process of parsing the statement database, some nouns have a specific naming category, so storing these nouns and their corresponding naming category to form a named entity recognition dictionary; the other characters and words cannot be recognized as a specific naming category, but they have specific rules, laws, and characteristics, so storing them to form a pattern rules database. For example: descriptions, misspelled characters correction, etc., where the descriptions comprise: color, shape, orientation, quantity, time, size, etc.; misspelled characters correction comprises: misspelled characters with words as the identification unit and correct words after correction.

In the preferred embodiment of the present invention, in order to effectively query the named entity recognition dictionary and the pattern rules database in the process of automatically labeling new report samples, and improve the accuracy of labeling, the step S2 further comprises: creating a prefix dictionary according to the named entity recognition dictionary, the prefix dictionary storing noun groups corresponding to each noun in the named entity recognition dictionary;

when the named entity recognition dictionary is composed of {d₁, . . . ,dᵢ, . . . ,dₙ}, any noun group in the prefix dictionary is expressed as: {d_{i_1}, . . . ,d_{i_j}, . . . ,d_{i_Li}}. Where, n denotes the total number of nouns in the named entity recognition dictionary, dᵢ denotes the i-th noun in the named entity recognition dictionary, i [symbol] 1, 2 . . . n, the i-th noun comprises Li characters arranged in sequence, d_{i_j} denotes the word consisting of the characters from the 1st one to the j-th one arranged in sequence, j [symbol] 1, 2 . . . Li;

traversing the prefix dictionary and keeping only one of the same words.

It can be understood that the nouns in the named entity recognition dictionary have relatively fixed meanings and rarely have ambiguities. Therefore, combined with common knowledge in the application field of the method, they can be easily obtained by parsing, that is, it is only needed to parse a few report samples to build a complete named entity recognition dictionary.

In order to label subsequent report samples more accurately, in a preferred embodiment of the present invention, when labeling subsequent unlabeled report samples using the named entity recognition dictionary and the pattern rules database, the prefix dictionary is used to accelerate the querying, and further, maximum matching principle and greedy matching principle are used to improve the accuracy of querying.

Accordingly, the step S3 specifically comprises: since the q-th report sample is collected, querying the named entity recognition dictionary, prefix dictionary and pattern rules database with the texts appearing in the report sample, to automatically label the current report sample.

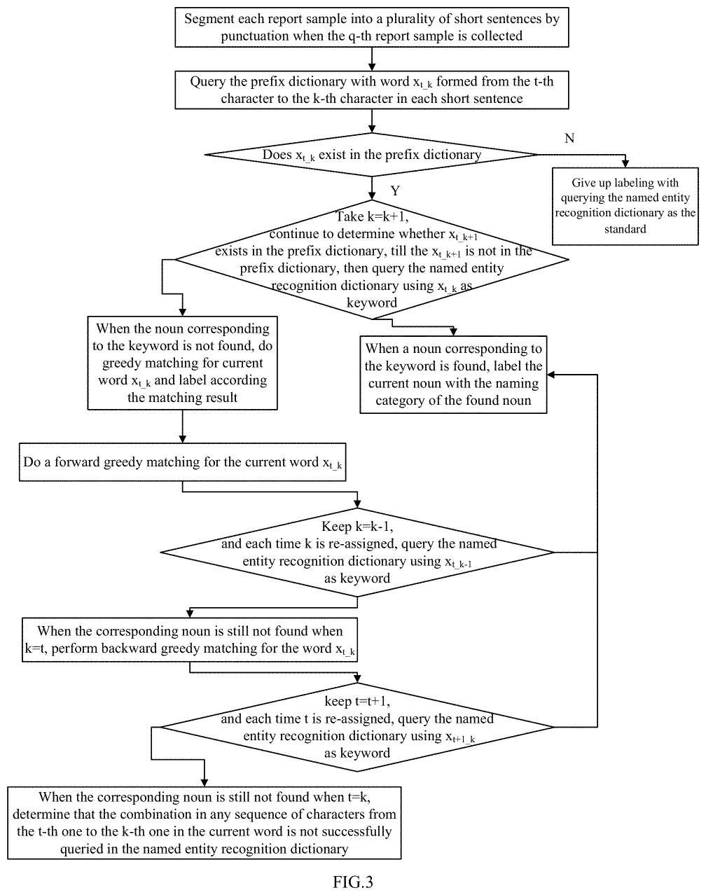

In the specific embodiment of the present invention, as shown in FIG. 3, step S3 specifically comprises: segmenting each report sample into a plurality of short sentences by punctuation when the q-th report sample is collected; querying the prefix dictionary with word x_{t_k} formed from the t-th character to the k-th character in each short sentence, the value of t is [1,XN], the value of k is [t,XN], where XN is the total number of characters in current short sentence; determining whether x_{t_k} exists in the prefix dictionary, taking t=1 for the first time of determination. If x_{t_k} exists in the prefix dictionary, taking k=k+1, continuing to determine if x_{t_k+1} exists in the prefix dictionary, till the x_{t_k+1} is not in the prefix dictionary, then querying the named entity recognition dictionary using x_{t_k} as the keyword, and if a noun corresponding to the keyword is found, labeling the current noun with the naming category of the found noun, and if the noun corresponding to the keyword is not found, doing greedy matching for current word x_{t_k} and labeling according the matching result; if the noun corresponding to the current word x_{t_k} is still not found by greedy matching, giving up labeling with querying the named entity recognition dictionary as the standard.

It should be noted that giving up labeling with querying the named entity recognition dictionary as the standard means that the current words can no longer be queried in the named entity recognition dictionary and labeled according to the result. In the preferred embodiment of the present invention, if it is determined that x_{t_k} does not exist in the prefix dictionary, continuing to use x_{t_k} to query the pattern rules database, where, if x_{t_k} exists in the pattern rules database, labeling according to the queried content, and if x_{t_k} does not exist in the pattern rules database, giving up labeling of x_{t_k}, and no further details are given here.

As above, the greedy matching comprises: doing a forward greedy matching for the current word x_{t_k}. In the process of forward greedy matching, keeping k=k−1, and each time k is re-assigned, querying the named entity recognition dictionary using x_{t_k-1} as keyword, and if the corresponding noun is found, labeling the current noun with the naming category of the found noun, and if the corresponding noun is still not found when k=t, performing backward greedy matching for the word x_{t_k}; in the process of backward greedy matching, keeping t=t+1, and each time t is re-assigned, querying the named entity recognition dictionary using x_{t+1_k} as keyword, and if the corresponding noun is found, labeling the current noun with the naming category of the found noun, and if the corresponding noun is still not found when t=k, determining that the combination in any sequence of characters from the t-th one to the k-th one in the current word is not successfully queried in the named entity recognition dictionary.

In turn, the labeling of all short sentences is completed to indirectly complete the labeling of the report samples.

For ease of understanding, the present invention describes a specific example for reference. For example, the noun recognition dictionary comprises nouns {“AB”, “ABCD”, “C”, “E”, “FEG”}, and each noun has a different naming category, then the prefix dictionary established is {“A”, “AB”, “ABC”, “ABCD”, “C”, “E”, “F”, “FE”, “FEG”}, where the prefixes “A” and “AB” of “ABCD” overlap with the prefix “A” and “AB” of the noun “AB” in the noun recognition dictionary, so the prefix dictionary keeps one for “A” and “AB”.

When labeling a new report sample, the short sentence queried is “ABCMFEX”. During querying the short sentence with the prefix dictionary in turn, as the t value increases, it is queried “ABC” in the prefix dictionary. Further, query the noun recognition dictionary using “ABC” as a keyword, and fail to find a specific noun. So, it is necessary to perform a greedy matching on “ABC”. In the process of forward greedy matching, keep k=k−1, that is, to query the noun recognition dictionary again with “AB” as the keyword. At this time, it can find “AB”, so label “AB” with its corresponding naming category. Then, continue to query the next character, and after specific querying, label “C” with its corresponding naming category. If “M” is not found, and not found in the pattern rules database, either, it can be labeled with a specific mark, such as “not appear”, “error”. When querying the prefix dictionary with “F”, it can be found. Continue to query the prefix dictionary with “FE”, and it can be found. Continue to query the prefix dictionary with “FEX”, but it fails. Query the noun recognition dictionary with “FE”, but it fails. Continue with greedy matching. During forward greedy matching, query the noun recognition dictionary with “F”, but it fails, and it fails to find in the pattern rule database either. Continue with backward greedy matching, and query the noun recognition dictionary with “E”. It can be found. Label “E” with its corresponding naming category, and label “F” before the “E” with a specific mark, such as “not appear”, “error”.

It should be noted that the description of the above method focuses on the querying of the named entity recognition dictionary, but the specific description, misspelled words, etc., due to their ambiguity, are not exhaustively listed. Therefore, the querying of the named entity recognition dictionary cannot be used, but the pattern rules database is used for querying. In particular, in the long-term application, the pattern rules database can be improved by using the pattern and rule characteristics to achieve more accurate labeling. In a specific example of the present invention, one of the rules in the pattern rules database is to use regular expressions to identify time and lesion size information, and to label them. For example: when recognizing the short sentence “A submucosal bulge with a size of about 0.3 cm is detected at proximal ileum”, it can label “0.3 cm” as “size” and “2 minutes and 25 seconds” as “time” according to this rule.

For step S4, an experienced doctor can provide assistance for review and verification. When there are errors or omissions in the labeling of the report samples, it means that the named entity recognition dictionary and the pattern rules database are not complete. At this time, the corrected report samples are inserted into the corpus database, and its associated databases and dictionaries are updated to make the next labeling more accurate. In this embodiment, although review with manual assistance is performed to improve labeling accuracy, in the review process, doctor only needs to verify the labeling results, with no need of a repeated labeling. Therefore, even if the review is manually assisted, it can still greatly save the time of manual labeling, and when the data in the corpus database is complete, the manual review is not needed.

Preferably, the present invention provides an electronic device comprising a memory and a processor. The memory stores a computer program that can run on the processor, and the processor executes the program to implement the steps of the method for labeling capsule endoscopy report described above.

Preferably, the present invention provides a computer-readable storage medium for storing a computer program. The computer program is executed by the processor to implement the steps of the method for labeling the capsule endoscopy report described above.

Those skilled in the art can clearly understand that, for the convenience and conciseness purposes, the specific working process of the electronic device and storable medium thereof described above cannot be repeated as it has been detailed in the foregoing method implementation.

In summary, the method, apparatus and medium for labeling capsule endoscopy report disclosed herein can build a database by parsing a small number of labeled report samples, making subsequent report samples query the database using specific rules, and then labeling the report samples automatically in a fast and effective manner. Further, the labeling results can be further verified through user-assisted check, and the corpus database can be updated according to the verification results, which can further improve the accuracy of labeling, greatly reduce the workload of users, save labor costs and improve labeling efficiency.

It should be understood that, although the specification is described in terms of embodiments, not every embodiment merely comprises an independent technical solution. Those skilled in the art should have the specification as a whole, and the technical solutions in each embodiment may also be combined as appropriate to form other embodiments that can be understood by those skilled in the art.

The present invention by no means is limited to the preferred embodiments described above. On the contrary, many modifications and variations are possible within the scope of the appended claims.

1. A method for labelling capsule endoscopy report, comprising:
collecting p report samples to establish an initial corpus database, any of the p report samples comprising an original text and labeled information, and the labeled information being a naming category corresponding to each noun in the original text;
parsing the report samples in the initial corpus database, to establish a named entity recognition dictionary and a pattern rules database, and removing duplicate texts from the named entity recognition dictionary and the pattern rules database;
wherein the named entity recognition dictionary comprises named categories in the report samples and nouns corresponding to each named category, and the pattern rules database comprises unrecognized texts in the report samples and rules, laws, and characteristics corresponding to the unrecognized texts;
since the q-th report sample is collected, q=p+1, querying the named entity recognition dictionary and the pattern rules database with texts appearing in the report sample, to automatically label the current report sample.

2. The method of claim 1, the method further comprising:
reviewing the automatically labeled report sample, revising errors when there are errors in the automatically labeled report sample, transferring the revised report sample to the original corpus database, and re-iterating and updating the named entity recognition dictionary and pattern rules database; identifying that the labelling of the current report sample completes when there are no errors in the automatically labeled report sample.

3. The method of claim 1, wherein the step “parsing the report samples in the initial corpus database” specifically comprises:
segmenting each report sample into a plurality of short sentences by punctuation and storing the first obtained short sentences to form a statement database.

4. The method of claim 3, wherein, in the process of establishing the statement database, the method further comprises:
parsing each obtained short sentence, and determining whether the current short sentence already exists in the statement database; omitting to process the current short sentence when the current short sentence already exists in the statement database, adding the current short sentence to the statement database when the current short sentence does not exist in the statement database;
parsing the statement database, to establish a named entity recognition dictionary and a pattern rules database, and removing duplicate texts from the named entity recognition dictionary and the pattern rules database.

5. The method of claim 1, wherein the step “parsing the report samples in the initial corpus database” further comprises:
creating a prefix dictionary according to the named entity recognition dictionary, the prefix dictionary storing noun groups corresponding to each noun in the named entity recognition dictionary;
when the named entity recognition dictionary is composed of {d₁, . . . ,dᵢ, . . . ,dₙ}, any noun group in the prefix dictionary is expressed as: {d_{i_1}, . . . ,d_{i_j}, . . . ,d_{i_Li}};
wherein, n denotes the total number of nouns in the named entity recognition dictionary, dᵢ denotes the i-th noun in the named entity recognition dictionary, i∈1, 2 . . . n, the i-th noun comprises Li characters arranged in sequence, d_{i_j} denotes the word consisting of the characters from the 1st one to the j-th one arranged in sequence, j∈1, 2 . . . Li;
traversing the prefix dictionary and keeping only one of the same words;
the step “automatically label the current report sample” specifically comprises: since the q-th report sample is collected, querying the named entity recognition dictionary, prefix dictionary and pattern rules database with the texts appearing in the report sample, to automatically label the current report sample.

6. The method of claim 5, wherein the step “automatically label the current report sample” further comprises:
segmenting each report sample into a plurality of short sentences by punctuation when the q-th report sample is collected;
querying the prefix dictionary with word x_{t_k} formed from the t-th character to the k-th character in each short sentence, the value of t is [1,XN], the value of k is [t,XN], wherein XN is the total number of characters in current short sentence;
determining whether x_{t_k} exists in the prefix dictionary, taking t=1 for the first time of determination,
taking k=k+1 when x_{t_k} exists in the prefix dictionary, continuing to determine whether x_{t_k+1} exists in the prefix dictionary, till the x_{t_k+1} is not in the prefix dictionary, then querying the named entity recognition dictionary using x_{t_k} as the keyword, and when a noun corresponding to the keyword is found, labeling the current noun with the naming category of the found noun, and when the noun corresponding to the keyword is not found, doing greedy matching for current word x_{t_k} and labelling according the matching result;
when the noun corresponding to the current word x_{t_k} is still not found by greedy matching, giving up labeling with querying the named entity recognition dictionary as the standard.

7. The method of claim 6, wherein “when the noun corresponding to the keyword is not found, doing greedy matching for current word x_{t_k} and labelling according the matching result” specifically comprises:
doing a forward greedy matching for the current word x_{t_k};
in the process of forward greedy matching, keeping k=k−1, and each time k is re-assigned, querying the named entity recognition dictionary using x_{t_k-1} as keyword, and when the corresponding noun is found, labelling the current noun with the naming category of the found noun, and when the corresponding noun is still not found when k=t, performing backward greedy matching for the word x_{t_k};
in the process of backward greedy matching, keeping t=t+1, and each time t is re-assigned, querying the named entity recognition dictionary using x_{t+1_k} as keyword, and when the corresponding noun is found, labelling the current noun with the naming category of the found noun, and when the corresponding noun is still not found when t=k, determining that the combination in any sequence of characters from the t-th one to the k-th one in the current word is not successfully queried in the named entity recognition dictionary.

8. The method of claim 1 wherein, in the process of querying the named entity recognition dictionary, prefix dictionary and pattern rules database with the texts appearing in the report sample, the method further comprises:
first querying the named entity recognition dictionary with the texts appearing in the report sample, and continuing to query the pattern rules database with the texts appearing in the report sample when no corresponding text is found in the named entity recognition dictionary.

9. An electronic apparatus, comprising a memory and a processor, wherein the memory stores computer programs that run on the processor,
and the processor executes the computer programs to implement the steps of a method for labelling capsule endoscopy report, wherein the method comprises:
collecting p report samples to establish an initial corpus database, any of the p report samples comprising an original text and labeled information, and the labeled information being a naming category corresponding to each noun in the original text
parsing the report samples in the initial corpus database, to establish a named entity recognition dictionary and a pattern rules database, and removing duplicate texts from the named entity recognition dictionary and the pattern rules database;
wherein the named entity recognition dictionary comprises named categories in the report samples and nouns corresponding to each named category, and the pattern rules database comprises unrecognized texts in the report samples and rules, laws, and characteristics corresponding to the unrecognized texts;
since the q-th report sample is collected, q=p+1, querying the named entity recognition dictionary and the pattern rules database with texts appearing in the report sample, to automatically label the current report sample.

10. A computer-readable storage medium storing computer programs,
wherein the computer programs are executed by the processor to implement the steps of a method for labelling capsule endoscopy report,
wherein the method comprises:
collecting p report samples to establish an initial corpus database, any of the p report samples comprising an original text and labeled information, and the labeled information being a naming category corresponding to each noun in the original text
parsing the report samples in the initial corpus database, to establish a named entity recognition dictionary and a pattern rules database, and removing duplicate texts from the named entity recognition dictionary and the pattern rules database;
wherein the named entity recognition dictionary comprises named categories in the report samples and nouns corresponding to each named category, and the pattern rules database comprises unrecognized texts in the report samples and rules, laws, and characteristics corresponding to the unrecognized texts;
since the q-th report sample is collected, q=p+1, querying the named entity recognition dictionary and the pattern rules database with texts appearing in the report sample, to automatically label the current report sample.
